(19) United States
(12) Patent Application Publication
(10) Pub. No.: US 2005/0154425 A1
(43) Pub. Date: Jul. 14, 2005

(54) Method and system to provide therapy for neuropsychiatric disorders and cognitive impairments using gradient magnetic pulses to the brain and pulsed electrical stimulation to vagus nerve(s)

(76) Inventors: Birinder R. Boveja, Milwaukee, WI (US); Angely Widhany, Milwaukee, WI (US)

Correspondence Address: Angely Widhany, P O Box 210095, Milwaukee, WI, 53221, US

(21) Appl. No.: 10/921,757

(22) Filed: Aug. 19, 2004

(51) Int. Cl. A61N 1/18

(52) U.S. Cl. 607/45

(57) ABSTRACT

A method and system of providing therapy or alleviating the symptoms of neuropsychiatric disorders and cognitive impairments comprises, providing gradient magnetic pulses to the brain and pulsed electrical stimulation to the vagus nerve(s) for afferent neuromodulation. These neuropsychiatric disorders and cognitive impairments include depression, bipolar depression, anxiety disorders, obsessive-compulsive disorders, schizophrenia, borderline personality disorders, sleep disorders, learning difficulties, memory impairments and the like. Gradient magnetic pulses are provided to the brain at approximately 1 KHz frequency in sessions that typically last for approximately 20 minutes, but can range from about 2 minutes to 5 hours. These gradient magnetic pulses produce a relatively constant electric field in the brain. Pulsed electrical stimulation to the vagus nerve(s) may be provided continuously in ON-OFF repeating cycles. The two stimulation therapies may be given in any order, any combination, or any sequence as determined by the physician. The two stimulation therapies may also be used with or without pharmaceutical therapy. Pulsed electrical vagus nerve stimulation (VNS) may be provided using an implanted pulse generator (IPG) or an external stimulator used in conjunction with an implanted stimulus-receiver. In one aspect of the invention the pulse generator system may comprise communication capabilities for networking over a wide area network, for remote interrogation and programming.

### FIELD OF INVENTION

This invention relates to providing electrical and magnetic pulses to the body, more specifically using combination of gradient magnetic pulses (GMP) to the brain, and pulsed electrical stimulation to vagus nerve(s) to provide therapy for neuropsychiatric disorders, and cognitive impairments.

### BACKGROUND

This disclosure is directed to method and system for providing adjunct (add-on) therapy for neuropsychiatric disorders and cognitive impairments, including depression, bipolar depression, anxiety disorders, obsessive-compulsive disorders, schizophrenia, borderline personality disorders, sleep disorders, learning difficulties, memory impairments and the like. The method and system comprises using combination of gradient magnetic pulses (GMP) to the brain and providing pulsed electrical stimulation to the vagus nerve(s) (VNS), to provide therapy. GMP and VNS may be used in combination to drug therapy, or as an alternative to drug therapy. The combination use of GMP and VNS is shown in conjunction with FIG. 1, and may be in any order, any combination or any sequence as determined by the physician. In the method of this application, the beneficial effects of GMP and VNS would be synergistic or at least additive. The rationale for the combined systems is that with GMP the electromagnetic energy is penetrated from outside to inside in a relatively uniform field, and with VNS the electrical pulses are delivered to the vagus nerve(s) 54. The afferent pulses resulting from vagus nerve stimulation travel to the Nucleus of Solitary Tract and eventually to other portions of the brain, via projections from the Nucleus of Solitary Tract (shown in FIGS. 4 and 5). This is described in more detail later.

### Background of Depression

Depression is a very common disorder that is often chronic or recurrent in nature. It is associated with significant adverse consequences for the patient, patient's family, and society. Among the consequences of depression are functional impairment, impaired family and social relationships, increased mortality from suicide and comorbid medical disorders, and patient and societal financial burdens. Depression is the fourth leading cause of worldwide disability and is expected to become the second leading cause by 2020.

Among the currently available treatment modalities include, pharmacotherapy with antidepressant drugs (ADDs), specific forms of psychotherapy, and electroconvulsive therapy (ECT). ADDs are the usual first line treatment for depression. Commonly the initial drug selected is a selective serotonin reuptake inhibitor (SSRI) such as fluoxetine (Prozac), or another of the newer ADDs such as venlafaxine (Effexor).

Several forms of psychotherapy are used to treat depression. Among these, there is good evidence for the efficacy of cognitive behavior therapy and interpersonal therapy, but these treatments are used less often than are ADDs. Phototherapy is an additional treatment option that may be appropriate monotherapy for mild cases of depression that exhibit a marked seasonal pattern.

Many patients do not respond to initial antidepressant treatment. Furthermore, many treatments used for patients who do not respond at all, or only respond partially to the first or second attempt at antidepressant therapy are poorly tolerated and/or are associated with significant toxicity. For example, tricyclic antidepressant drugs often cause anticholinergic effects and weight gain leading to premature discontinuation of therapy, and they can by lethal in overdose (a significant problem in depressed patients). Lithium is the augmentation strategy with the best published evidence of efficacy (although there are few published studies documenting long-term effectiveness), but lithium has a narrow therapeutic index that makes it difficult to administer; among the risks associated with lithium are renal and thyroid toxicity. Monoamine oxidase inhibitors are prone to produce an interaction with certain common foods that results in hypertensive crises. Even selective serotonin reuptake inhibitors can rarely produce fatal reaction in the form of a serotonin syndrome.

Physicians usually reserve ECT for treatment-resistant cases or when they determine a rapid response to treatment is desirable. ECT is also associated with significant risks: long-lasting cognitive impairment following ECT significantly limits the acceptability of ECT as a long-term treatment for depression. Therefore, there is a compelling unmet need for non-pharmacological well-tolerated and effective long-term or maintenance treatments for patients who do not respond fully, or for patients who do not sustain a response to first-line pharmacological therapies.

FIG. 2 (shown in table form) generally highlights some of the advantages and disadvantages of various forms of nonpharmalogical interventions for the treatment of depression. For example, deep brain stimulation is regionally very specific which is good, but on the other hand requires very invasive surgical procedure. As another example, ECT has clinical applicability in the short run, but on the other hand is associated with long-lasting cognitive impairments. Considering the advantages and disadvantages of different existing treatments, as shown in conjunction with FIG. 2, a combination of GMP therapy which involves low level magnetic fields and vagus nerve stimulation is an ideal combination for device based interventions, with or without concomitant drug therapy. Furthermore, in this unique combination, GMP induces stimulation from outside, and vagus nerve stimulation (VNS) approaches the stimulation from inside the brain, as shown in conjunction with FIG. 1. The initiation and delivery of these two interventions may be in any sequence or combination, and may be in addition to any drug therapy. For example, a patient implanted with vagal nerve stimulator may be started on GMP therapy, or alternatively a patient receiving GMP may be implanted with a vagus nerve stimulator. Of course, this may be in addition to any drug therapy that may be given to a patient.

In some patients the beneficial effects of GMP may last for sometime. These patient's may be implanted with the nerve stimulator sometime after receiving their last dose of GMP therapy. This form of combination therapy, where a patient receives GMP therapy initially and sometime later receives pulsed electrical stimulation therapy, is also considered within the scope of the invention.

### PRIOR ART

U.S. Pat. No. 5,879,299 (Posse) et al. is generally directed to method and system for providing prelocalization of a volume of interest and for rapidly acquiring a data set for generating spectroscopic images. Spectroscopic imaging data is acquired by an echo planar spatial-spectral imaging sequence in which the gradient reversal frequency is a integer factor of n greater than the gradient reversal frequency required to sample the spectral width. There is no disclosure or suggestion for providing any kind of therapy for neruopsychiatric disorders.

U.S. Pat. No. 6,572,528 B2 (Rohan et al.) and U.S. Patent Application No. U.S. 2004/0010177 A1 (Rohan et al.) is generally directed to magnetic field stimulation techniques. There is no disclosure or even suggestion for combining magnetic fields to the brain with electrical pulses to the vagus nerve to provide therapy for neuropsychiatric disorders.

U.S. Pat. No. 5,270,654 (Feinberg et al.) is generally directed to fast magnetic resonance imaging using combined gradient echoes and spin echoes. Again, there is no disclosure or suggestion for providing any kind of therapy for neruopsychiatric disorders.

U.S. Pat. No. 6,472,871 B2 (Ryner) is generally directed to generating a spectroscopic image using magnetic resonance for obtaining spectroscopic data from voxels by subjecting the sample to repeated magnetic resonance experiments.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) and U.S. Pat. No. 6,356,788 B2 (Boveja) are directed to adjunct therapy for neurological and neuropsychiatric disorders using an implanted lead-receiver and an external stimulator.

### Other Publications

Rohan M. et al., “Low-Field Magnetic Stimulation in Bipolar Depression Using an MRI-Based stimulator”. American Journal of Psychiatry, vol. 161: pp. 93-98, 2004.

### SUMMARY OF THE INVENTION

A novel method for providing therapy or alleviating the symptoms of neuropsychiatric disorders and cognitive impairments comprises, providing gradient magnetic pulses (GMP) to the brain and afferent neuromodulation of the vagus nerve(s) (VN) with pulsed electrical stimulation. The combination of GMP and VN stimulation provides a more ideal combination for device based interventions, with or without concomitant drug therapy. In this novel method of therapy, GMP induces stimulation from the outside, and selective vagus nerve stimulation approaches the stimulation from inside the brain.

Accordingly in one aspect of the invention, method and system to provide therapy for or alleviate the symptoms of neuropsychiatric disorders and cognitive impairments comprises providing gradient magnetic pulses to the brain of a patient and afferent neuromodulation of a vagus nerve(s) with electrical pulses.

In another aspect of the invention, the combination of gradient magnetic pulses provided to the brain and electrical pulses provided to vagus nerve(s) are in any sequence or any combination, as determined by the physician.

In another aspect of the invention, the gradient magnetic pulses have a frequency of about 1 kHz and produce electric fields of the same frequency.

In another aspect of the invention, the gradient magnetic pulses to the brain can be provided by an echo-planer magnetic resonance spectroscopic imaging (EP-MRSI) system, among other systems.

In another aspect of the invention, the gradient magnetic pulses induce relatively uniform electric fields in the brain with an amplitude of between 1 V/m and 100 V/m.

In another aspect of the invention, the afferent modulation of the vagus nerve(s) is by providing electric pulses at any point along the length said vagus nerve(s).

In another aspect of the invention, the vagus nerve(s) is/are neuromodulated bilaterally.

In another aspect of the invention, the system to provide electrical pulses to the vagus nerve(s) has both implanted and external components, and may be one selected from the following group: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a programmable implantable pulse generator (IPG); e) a combination implantable device comprising both a stimulus-receiver and a programmable IPG; and f) an IPG comprising a rechargeable battery.

In yet another aspect of the invention, the system for providing electrical pulses to the vagus nerve(s) can be remotely interrogated or remotely programmed over a wide area network, either wirelessly or over land-lines.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram depicting the concept of the invention, where a patient receives gradient magnetic pulses to the brain, and pulsed electrical stimulation to vagus nerve(s).

FIG. 2 depicts in table form, the peculiarities of different forms of device based therapies for neuropsychiatric disorders.

FIG. 3 is a diagram showing the overall structure of the brain.

FIG. 4 is a schematic diagram of the brain showing relationship of vagus nerve and solitary tract nucleus to other centers of the brain.

FIG. 5 is a simplified block diagram illustrating the connections of solitary tract nucleus to other centers of the brain.

FIG. 6 is a diagram showing a prior art method for delivering gradient magnetic pulses.

FIG. 7 is a diagram depicting methodology for providing gradient magnetic pulses to the brain of a patient.

FIG. 8 is a diagram showing the morphology of gradient magnetic pulses and the resulting electrical pulses.

FIG. 9 depicts the relatively uniform fields of gradient magnetic pulses.

FIG. 10 depicts the relatively non-uniform field as supplied with the technique of repetitive transcranial magnetic stimulation (rTMS).

FIG. 11 depicts a cut away section of the brain, showing the corpus callosum, which connects the right and left hemispheres of the brain.

FIG. 12A shows the pulse train transmitted to the vagus nerve.

FIG. 12B shows the ramp-up and ramp-down characteristic of the pulse train.

FIG. 13 is a simplified block diagram depicting supplying amplitude and pulse width modulated electromagnetic pulses to an implanted coil.

FIG. 14 depicts a customized garment for placing an external coil to be in close proximity to an implanted coil.

FIG. 15 shows coupling of the external stimulator and the implanted stimulus-receiver.

FIG. 16 is a schematic diagram of an implantable lead.

FIG. 17 is a schematic diagram showing the implantable lead and one form of stimulus-receiver.

FIG. 18 is a schematic block diagram showing a system for neuromodulation of the vagus nerve, with an implanted component which is both RF coupled and contains a high value capacitor for power source.

FIG. 19 is a simplified block diagram showing control of the implantable neurostimulator with a magnet.

FIG. 20 is a schematic diagram showing implementation of a multi-state converter.

FIG. 21 is a simplified block diagram of an implantable pulse generator.

FIG. 22 is a functional block diagram of a microprocessor-based implantable pulse generator.

FIG. 23 shows details of implanted pulse generator.

FIG. 24A is a diagram showing the two modules of the implanted pulse generator (IPG).

FIG. 24B is a diagram with a coil outside of the titanium can.

FIG. 25 is a schematic and functional block diagram showing the components and their relationships to the implantable pulse generator/stimulus-receiver.

FIG. 26A shows a picture of the combination implantable stimulator.

FIG. 26B shows assembly features of the implantable portion of a vagus nerve stimulation system.

FIG. 27 depicts an embodiment where the implantable system is used as an implantable, rechargeable system.

FIG. 28 depicts remote monitoring of stimulation devices.

FIGS. 29 is a simplified diagram showing communication of modified PDA/phone, with an external stimulator via a cellular tower/base station.

FIG. 30 is a simplified block diagram of the networking interface board.

### DETAILED DESCRIPTION OF THE INVENTION

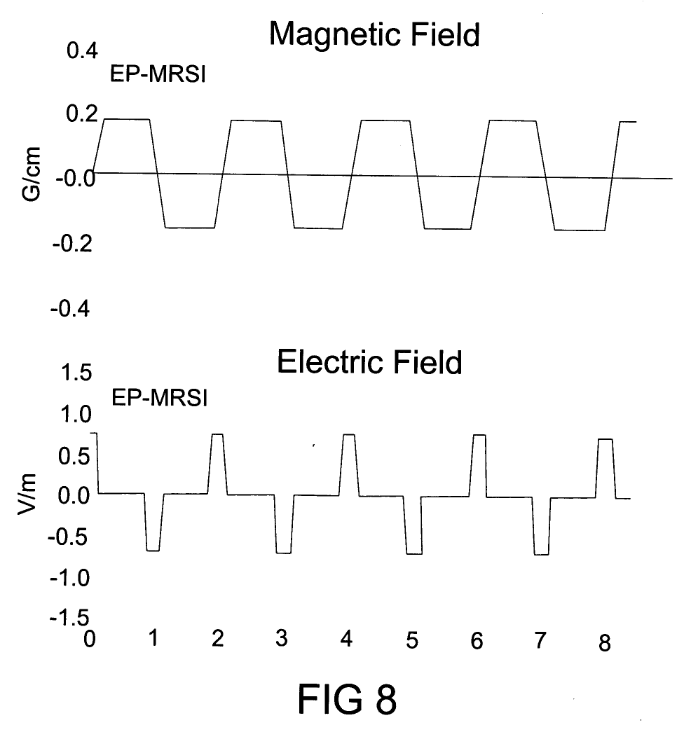

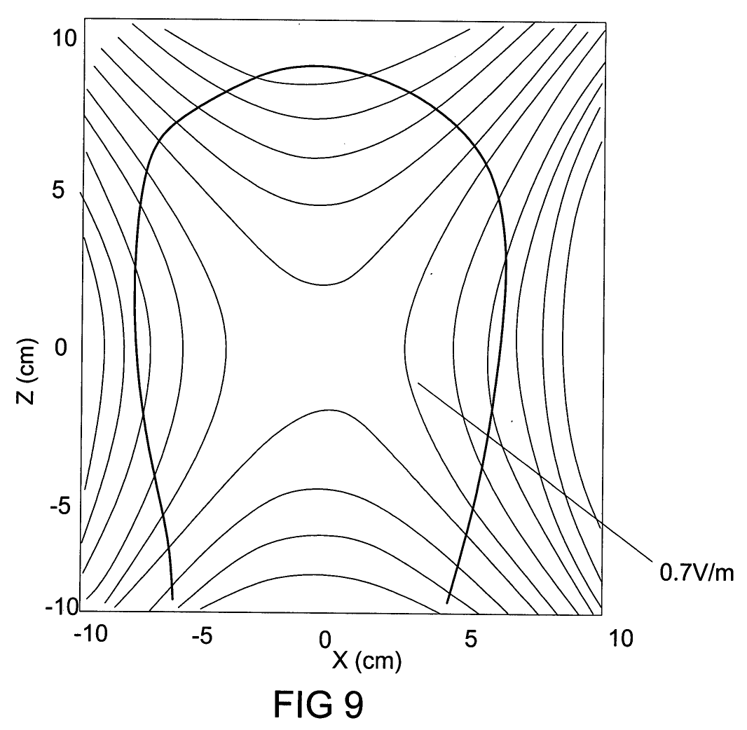

In the method and system of this invention, magnetic and electric fields are applied to the whole brain and electrical pulses are delivered to the vagus nerve(s), for treating or alleviating the symptoms of neuropsychiatric disorders and cognitive impairments. These disorders include depression, bipolar depression, anxiety disorders, obsessive-compulsive disorders, schizophrenia, borderline personality disorders, sleep disorders, learning difficulties, memory impairments and the like. This stimulation therapy may be used as adjunct (add-on) therapy. The magnetic and electric fields to the whole brain may be supplied using an echo-planer magnetic resonance spectroscopic imaging (EP-MRSI) device, or any other appropriate device for delivering gradient magnetic pulses of appropriate characteristics. Pulsed electrical stimulation to the vagus nerve(s) 54 is supplied using a pulse generator means and a lead with electrodes in contact with nerve tissue. The two stimulation therapies may be applied in any combination or sequence. The whole brain magnetic and electric fields (FIGS. 8 and 9) are typically applied for approximately 20 minutes using gradient magnetic pulses (GMP). Vagus nerve stimulation is typically applied 24 hours/day, 7 days a week, in repeating cycles. The time periods of either GMP or VNS may vary by any amount at the discretion of the physician.

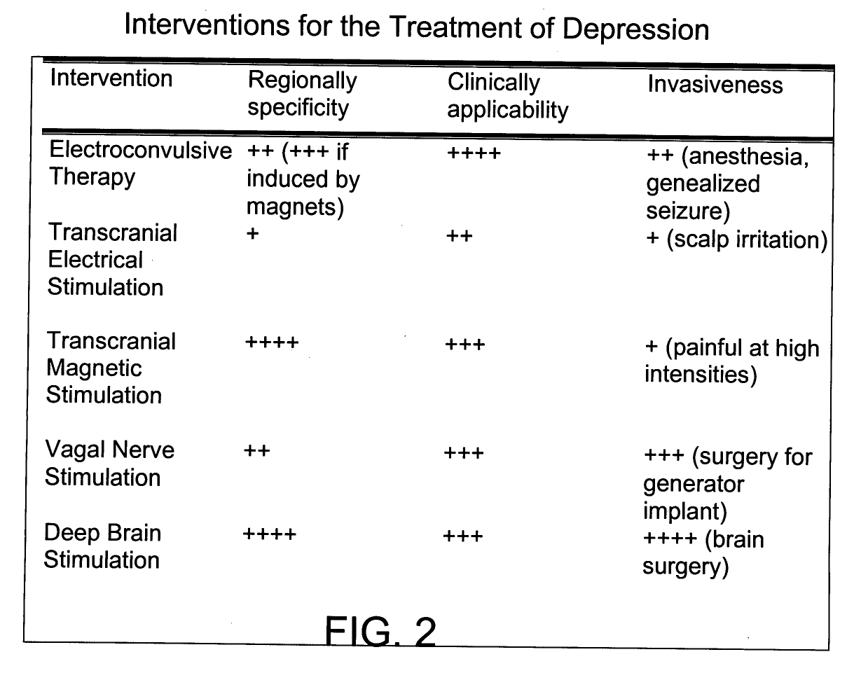

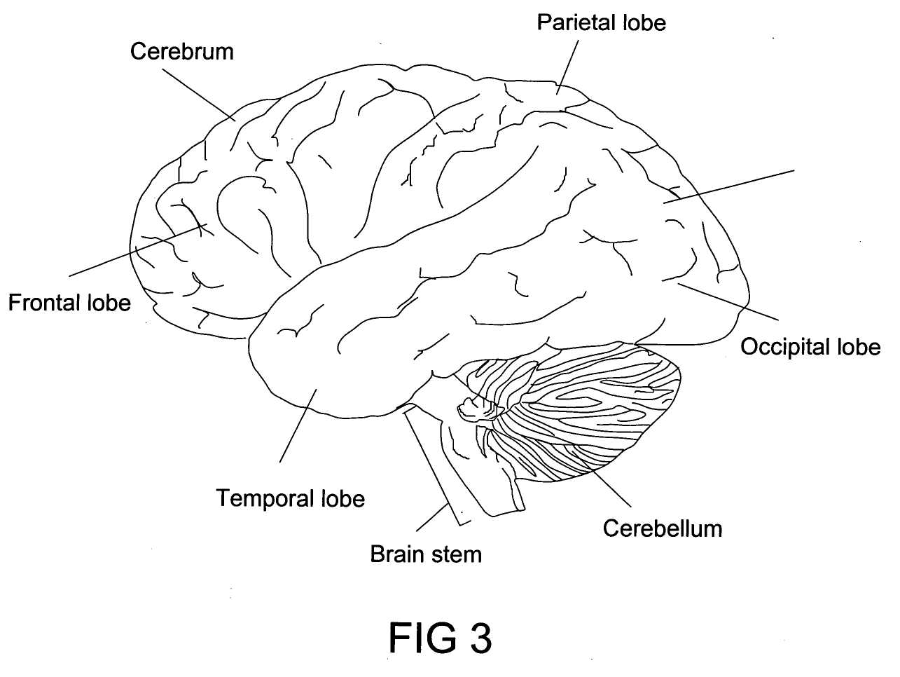

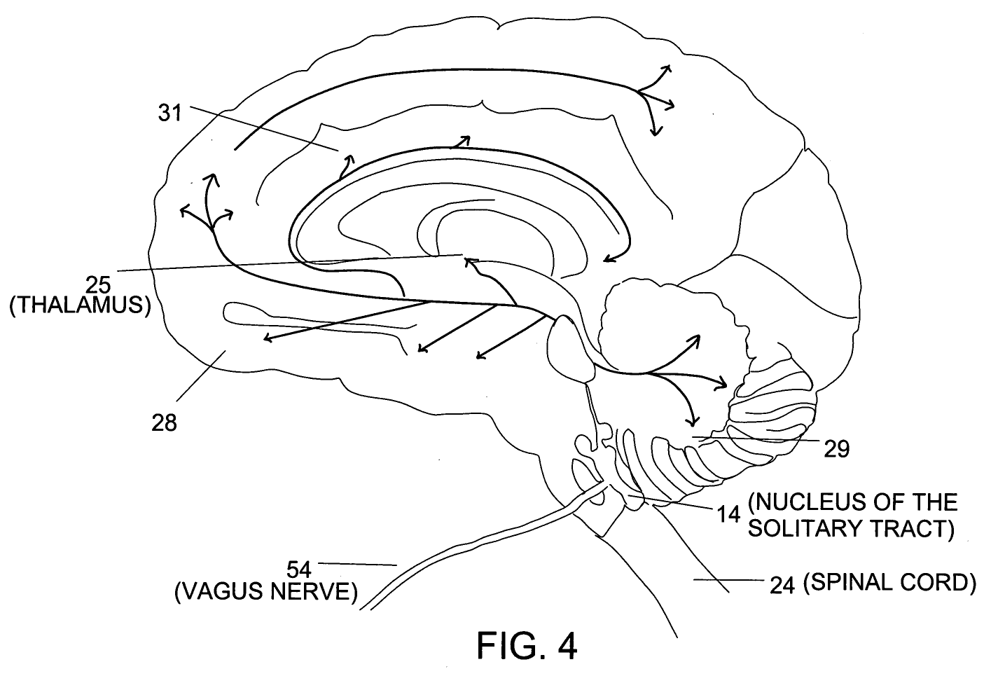

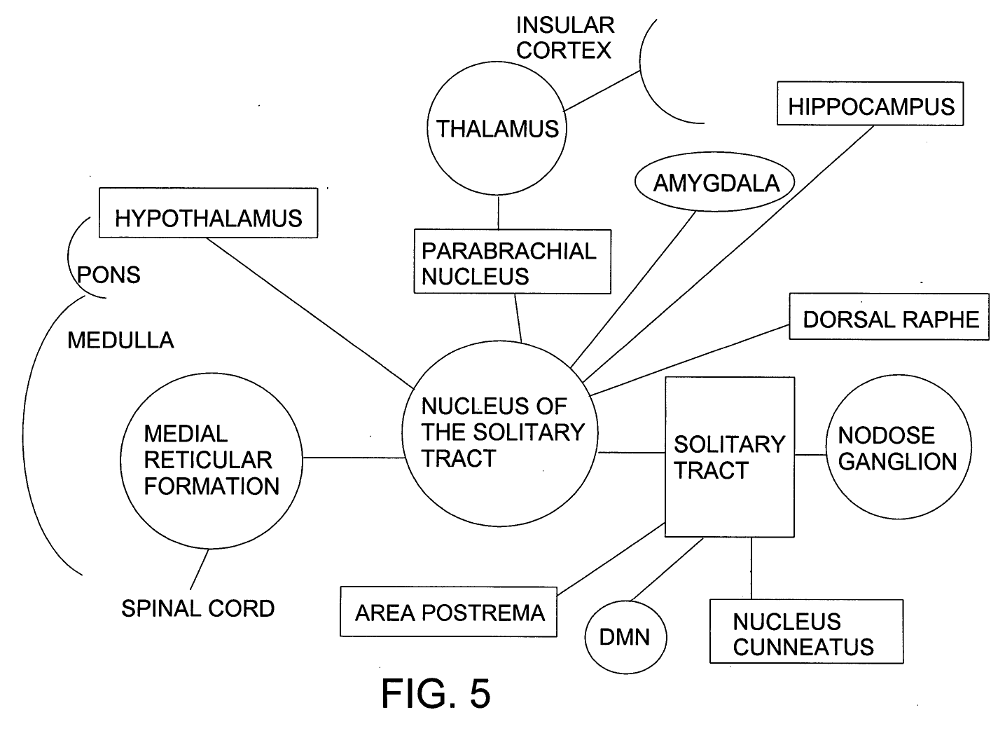

Advantageously, the two types of stimulations approach the relevant centers in the brain via different approaches. With GMP the approach is via uniformly distributed magnetic fields leading to electrical fields from the outside, and with vagus nerve(s) 54 pulsed electrical stimulation, the approach to centers in the brain is from the inside (FIG. 4). Shown in conjunction with FIG. 3, which is an overall diagram of the brain, and in conjunction with FIGS. 4 and 5, afferent electrical neuromodulation of the vagus nerve(s) reaches the centers in the brain via projection from the Nucleus of the Solitary Tract (FIG. 5). Further, shown in conjunction with FIG. 2 the efficacy and invasiveness of the two stimulation therapies are also matched to provide the patient with balanced risk/benefit ratio. GMP typically provides immediate benefits of mood improvement and no known side effects, but the benefits may or may not be very long lasting. With VNS the time profile of anti-depressant benefits are sustained over a long period of time, even though they may be slow to accumulate. Therefore, advantageously the combined benefits are both immediate and long lasting, providing a more ideal therapy profile, and cover a broader spectrum of patient population.

As mentioned previously, any combination or sequence of these two energies may be applied, and is determined by the physician for each patient.

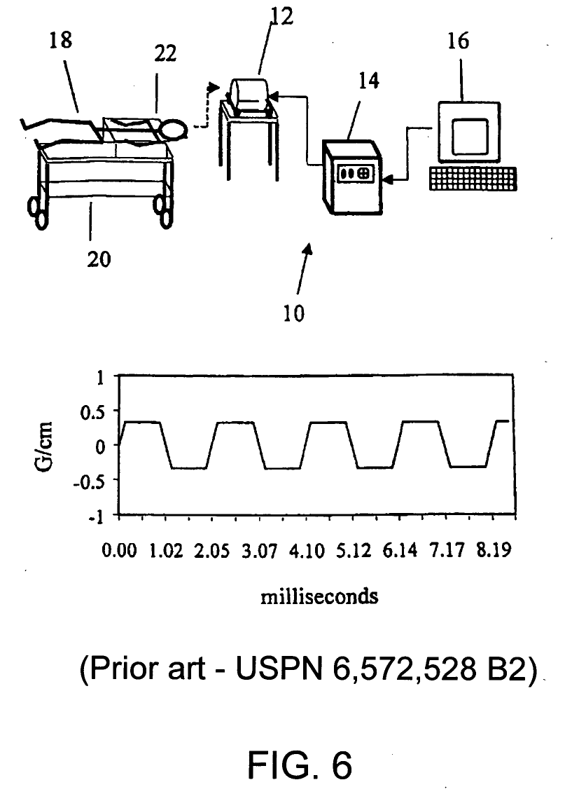

One prior art (U.S. Pat. No. 6,572,528 B2) system for providing gradient magnetic pulses is shown in conjunction with FIG. 6. For the purposes of the current invention, gradient magnetic pulses (GMP) may be provided using the system disclosed in this patent, and is incorporated herein by reference. Alternatively, other systems such as available from General Electric (GE) Corporation (Wisconsin, USA) may be used. Regardless of which system is used, the magnetic field induces an electric field in the patient's brain. The general relationship between magnetic field parameters and the electric field is described by Maxwell's equation as stated below, (more details are found in any appropriate Physics textbook).

∇xE(x, y, z, t)=−∂B(x, y, z, t)/∂t, where ∇xE is the curl of the electric field, and ∂B/∂t is the rate of change of the magnetic field over time. In Cartesian coordinates, this equation becomes:
∂Eₓ/∂y−∂E_{y}/∂x=−∂B_{z}/∂t,
∂E_{y}/∂z−∂E_{z}/∂y=−∂Bₓ/∂t,
∂E_{z}/∂x−∂Eₓ/∂x=−∂Bₓ/∂t,

One techniques to deliver gradient magnetic pulses is magnetic resonance spectroscopic imaging (MRSI). This technique is incorporated in this application and is one method to provide gradient magnetic pulses in one embodiment. Other systems in development, or developed in the future to provide gradient magnetic pulses can also be used in conjunction with VNS therapy for the purpose of this invention, and are within the scope of this invention.

Spectroscopic imaging techniques have been developed which combine magnetic resonance imaging (MRI) techniques with nuclear magnetic resonance (NMR) spectroscopic techniques, thus providing a spatial image of the chemical composition. There has been increasing interest in the study of brain metabolism using proton MR spectroscopy and spectroscopic imaging because of its noninvasive assessment of regional biochemistry.

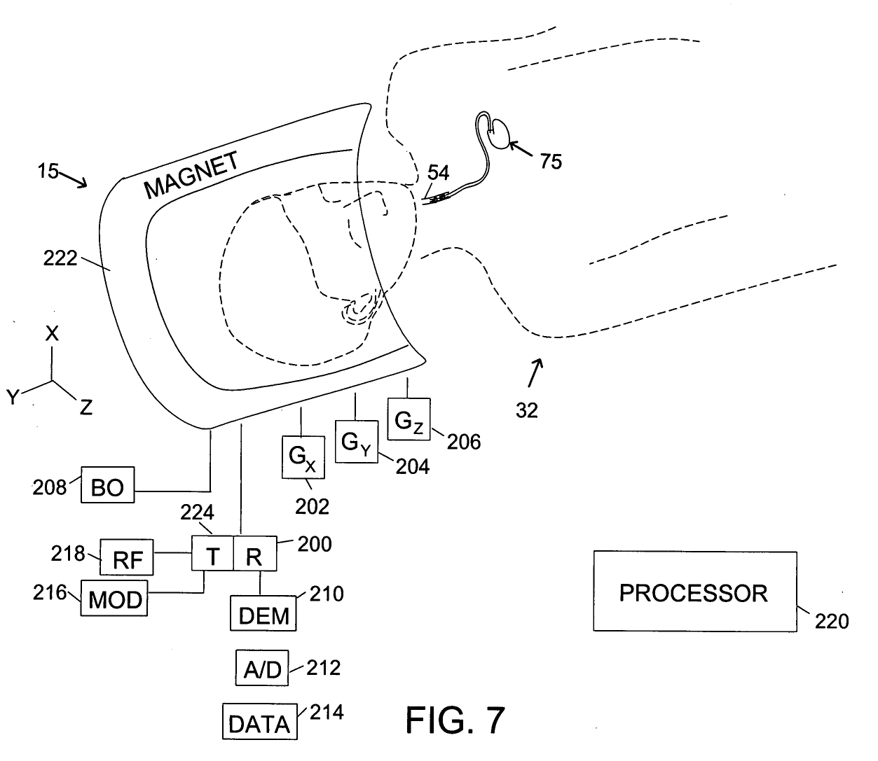

Shown in conjunction with FIG. 7 is a block diagram for an in-vivo NMR imaging system which is capable of providing gradient magnetic pulses to a patient's head. The system includes a magnet 222 for generating a large static magnetic field. The magnet is sufficiently large and has a bore such that the magnet goes over the patient's head and surrounds it. The patient's head is positioned and the magnetic field is generated by a magnetic field generator indicated at 208 by block Bₒ. Radiofrequency (RF) pulses are generated utilizing RF generator 218, and the RF pulses are shaped using modulator 216. The shape of a modulated pulse could be any predetermined shape, and for example may be Gaussian or Sinc (i.e., sin (bt)/bt, where b is a constant, and t is time). Shaped pulses are usually employed in order to shape and limit the bandwidth of the pulse, thereby restricting excitation by the RF pulse to spins that have Larmor frequencies within the RF pulse band-width. A RF pulse signal is transmitted to coils in the magnet assembly which are not shown. The coils may be surface coils, or heat coils for example. The duration and amplitude of the RF pulse determine the amount which the net magnetization is “tipped”. Tip angles of substantially 90° are employed for a stimulated echo pulse sequence.

Gradient generators 202, 204, and 206, which include respective gradient coils, produce the Gₓ, G_{y}, and G_{z} magnetic fields in the direction of the polarizing magnetic field Bₒ, but with gradients directed in the x, y, and z directions, respectively. The use of the Gₓ, G_{y}, and G_{z} are well known in the art, including such uses as dephasing or rephasing excited spins, spatial phase encoding or spatial gradient encoding acquired signals, and spatial encoding of the Larmor frequency of nuclei for slice selection. Induced nuclear magnetic resonance signals are detected by receiver coils in the magnet (not shown). The receiver coils and the transmitter coils may be the same, with a transmit/receive (T/R) switch being used to select transmission or reception of radio frequency signals to or from the coils, respectively. The received signal is demodulated by demodulator 210, and the demodulated signal is amplified and processed in the analog-to-digital processing unit 212 to provide data as indicated at 214. The entire process is monitored and controlled by the processor means 220 which, according to the functional block diagram of FIG. 7 and to the components found in known commercial or experimental systems that are used to control and monitor the entire process, includes components necessary to control the timing, amplitudes and shapes of the control signals for the various elements of the MRI system and typically includes programming, computing, and interfacing means.

Gradient magnetic energy is typically applied for approximately 20 minutes per session, but may vary at the discretion of the physician. EP-MRSI employs oscillating magnetic fields that are similar to those used in functional magnetic resonance imaging (fMRI) but that differ from the usual fMRI scan in field direction, waveform frequency, and strength. The characteristics of the electromagnetic fields of EP-MRSI can be further illustrated by comparing the fields of EP-MRSI with those of well known repetitive transcranial magnetic stimulation (rTMS). EP-MRSI and rTMS both subject the brain to time-varying magnetic and electric fields. The fields in the EP-MRSI are very different from those in rTMS in strength, uniformity, direction, and timing. It is noteworthy that the EP-MRSI fields are 100 to 1,000 times weaker than the rTMS fields, penetrate throughout the whole brain, and are delivered at 1 kHz. The EP-MRSI magnetic field of interest is the readout gradient. This magnetic field is delivered in a series of 512 trapezoid pulses that are each 1 msec long, as shown in FIG. 8. The series of 512 pulses is repeated every 2 seconds for 128 repetitions (4 minutes) for each scan. The magnetic field is an MRI gradient field with the form of a linear ramp, with a zero field in the middle of the coil and a ramp of 0.3 gauss/cm (G/cm) that reaches a maximum of less than 10 G in the brain. Also shown in conjunction with FIG. 8, the electric field for EP-MRSI consists of a series of alternating square pulses that are each about 0.25 msec long and that also occur at 1 kHz. This waveform is shown in bottom part of FIG. 8. The electric field is constant during each pulse. The strength of the electric field is about 0.7 V/m, is uniform to 5%, and is in the direction of the subject's right to left. A contour plot of the electric field magnitude is shown in FIG. 9.

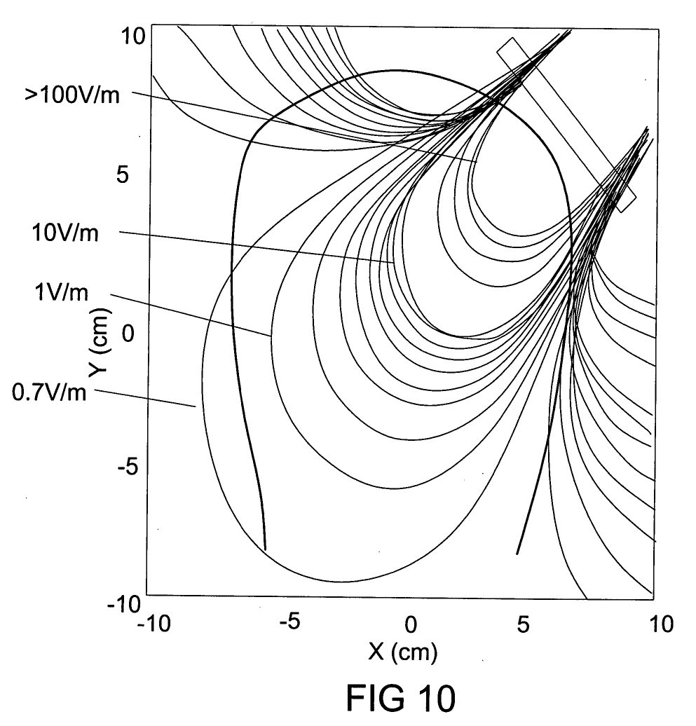

In contrast, shown in conjunction with FIG. 10 of a contour plot, the fields in rTMS are produced by a small coil some inches across, and are large and nonuniform. The rTMS magnetic field is delivered in single-cycle sine pulses with a period of about 0.28 msec at 1-20 Hz for 20 minutes. rTMS magnetic fields have strengths up to 2 T (20,000 G) at locations in the cortex falling off to less than 10 G at a distance of 20 cm away. The rTMS field consists of single-cycle cosine pulses with the same 0.28-msec period, at 1-20 Hz, similar to the magnetic field pulses. The electric field reverses sign during each pulse. The strength of the rTMS electric field ranges from more than 500 V/m in the cortex under the coil to 1 V/m 20 cm away. In contrast to EP-MRSI, this electric field is highly nonuniform, and it has no well-defined direction in the brain. In the contour plot of the rTMS electric field strength (FIG. 10), it is noteworthy that the distribution of the rTMS field in the head depends greatly on the position of the coil; for EP-MRSI, head position is less significant.

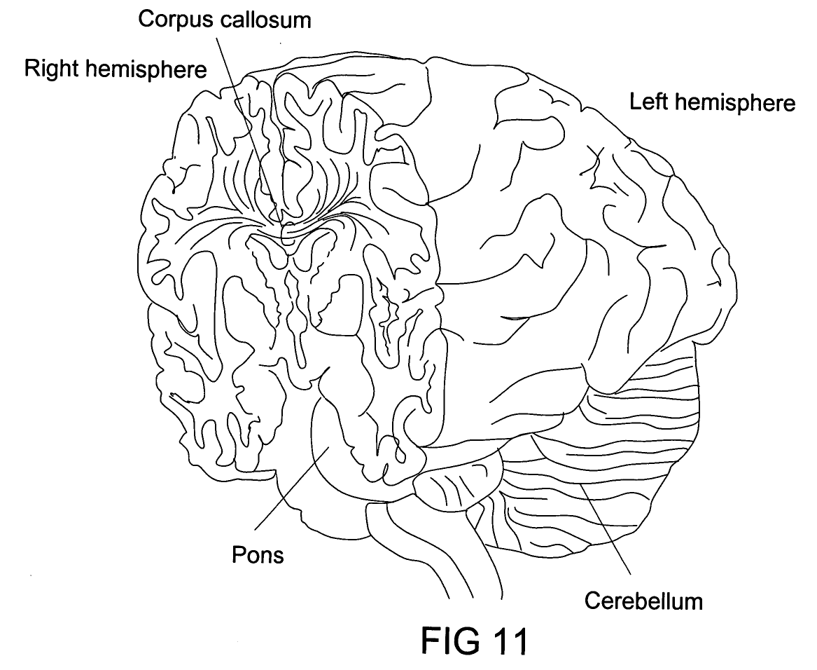

The uniformity, unidirectionality, and whole-brain penetration of the EP-MRSI treatment may be selecting very different structures in the brain, compared with the well known rTMS. It is hypothesized that the right-to-left electric fields in EP-MRSI could be selecting corpus callosum, whose axons lie in that direction. The corpus callosum is a broad band of neurons connecting the right and left hemispheres, and is shown in FIG. 11. Given that neuronal conduction processes occur on millisecond time scales, it is believed that the monophasic pulses delivered at 1 KHz in the EP-MRSI system, which are on the same time scale as neuronal processed, may interact with these processes, particularly with conduction processes that have time constants greater than 1 msec.

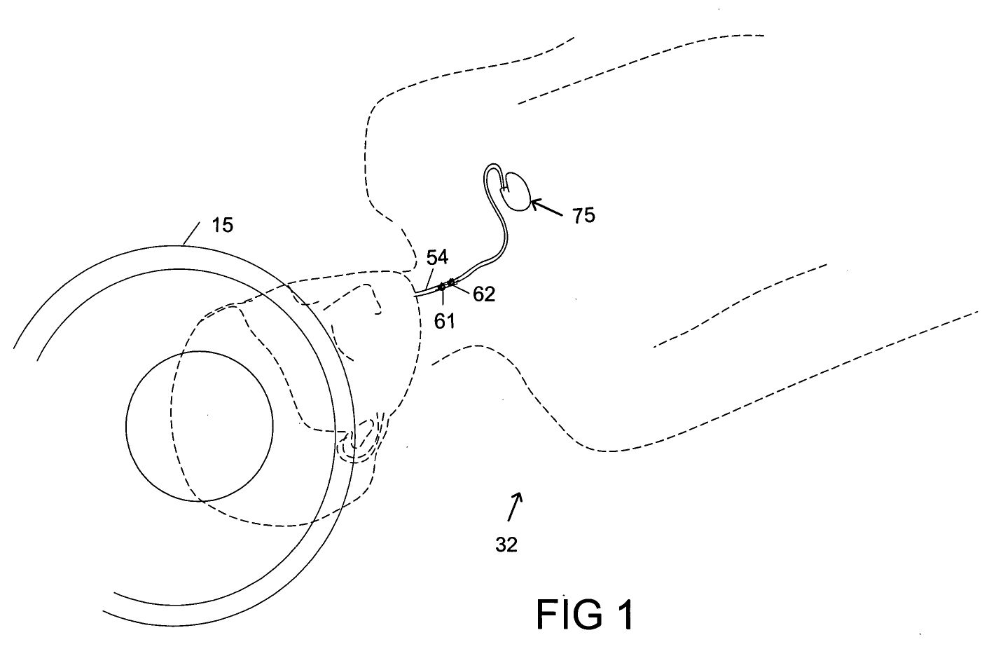

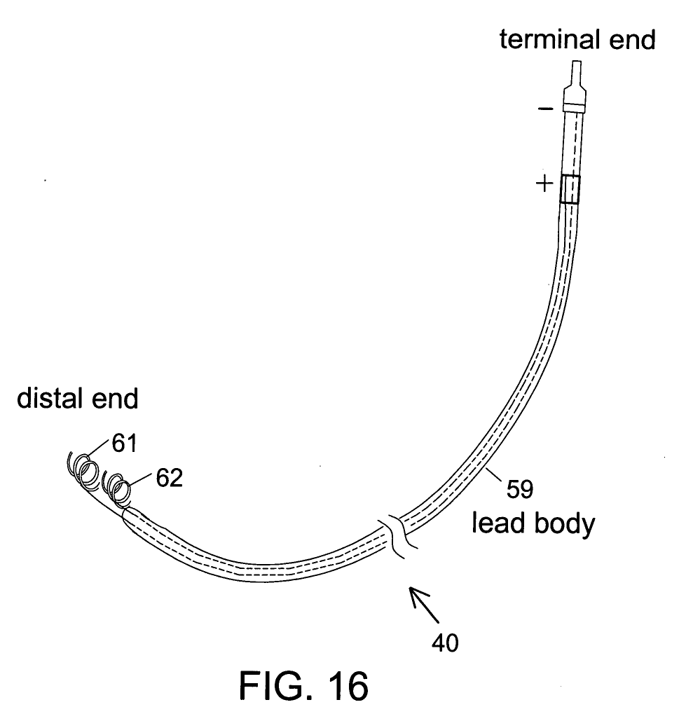

As shown in conjunction with FIG. 1, pulsed electrical stimulation to the vagus nerve(s) 54 is provided utilizing a pulse generator means and an implanted lead 40. The implanted lead comprises a pair of electrodes 61, 62 (FIG. 16) that are adapted to be in contact with the vagus nerve(s) 54 for directly stimulating the nerve tissue. These electrodes may be placed on the vagus nerve at around the neck level or around the diaphragmatic level, either just above or below the diaphragm. Also the electrodes may be implanted on one nerve for unilateral stimulation, or on both nerves for bilateral stimulation. The terminal end of the lead connects to either a pulse generator or a stimulus-receiver means.

Electrical pulses are provided to the vagus nerve(s) 54 using a system that comprises both implantable and external components. The system to provide selective stimulation may be selected from one of the following:
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a programmable implantable pulse generator (IPG);
e) a combination implantable device comprising both a stimulus-receiver and a programmable IPG; and
f) an IPG comprising a rechargeable battery.

The pulse generator means is in electrical contact with a lead, which is adapted to be in contact with the vagus nerve(s) or its branches. The pulse generator/stimulator can be of any form or type including those that are in current use or in development or to be developed in future. U.S. Pat. Nos. 4,702,254, 5,025,807, and 5,154,172 (Zabara) describe pulse generator and associated software to provide VNS therapy which are also included here by reference, in this invention for application of VNS.

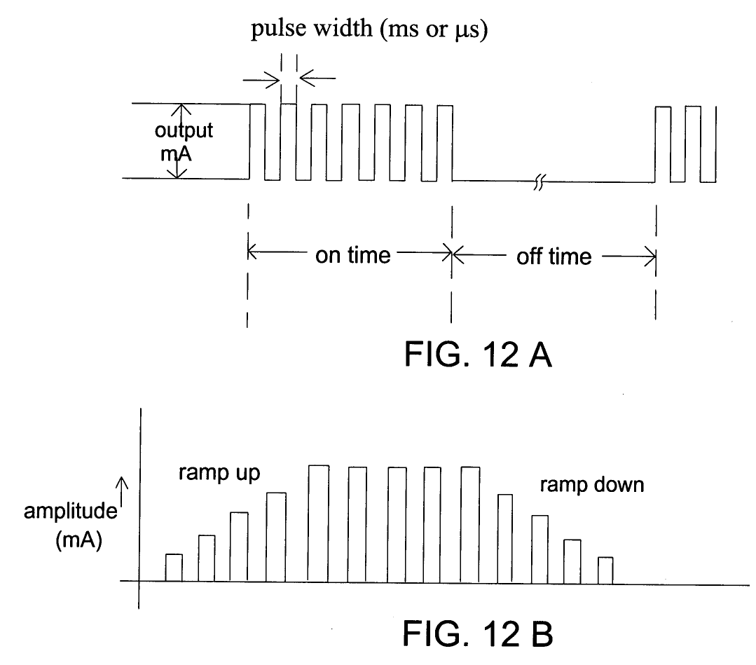

Using any of these systems, selective pulsed electrical stimulation is applied to vagus nerve(s) for afferent neuromodulation, at any point along the length of the nerve. The waveform of electrical pulses is shown in FIG. 12A. As shown in FIG. 12B, for patient comfort when the electrical stimulation is turned on, the electrical stimulation is ramped up and ramped down, instead of abrupt delivery of electrical pulses.

These stimulation systems for vagus nerve modulation are more fully described in a co-pending application (Ser. No. 10/841,995), but are mentioned here briefly for convenience. In each case, an implantalbe lead is surgically implanted in the patient 32. The vagus nerve(s) is/are surgically exposed and isolated. The electrodes on the distal end of the lead are wrapped around the vagus nerve(s) 54, and the lead is tunneled subcutaneously. A pulse generator means is connected to the proximal end of the lead. The power source may be external, implantable, or a combination device.

### Implanted Stimulus-Receiver with an External Stimulator

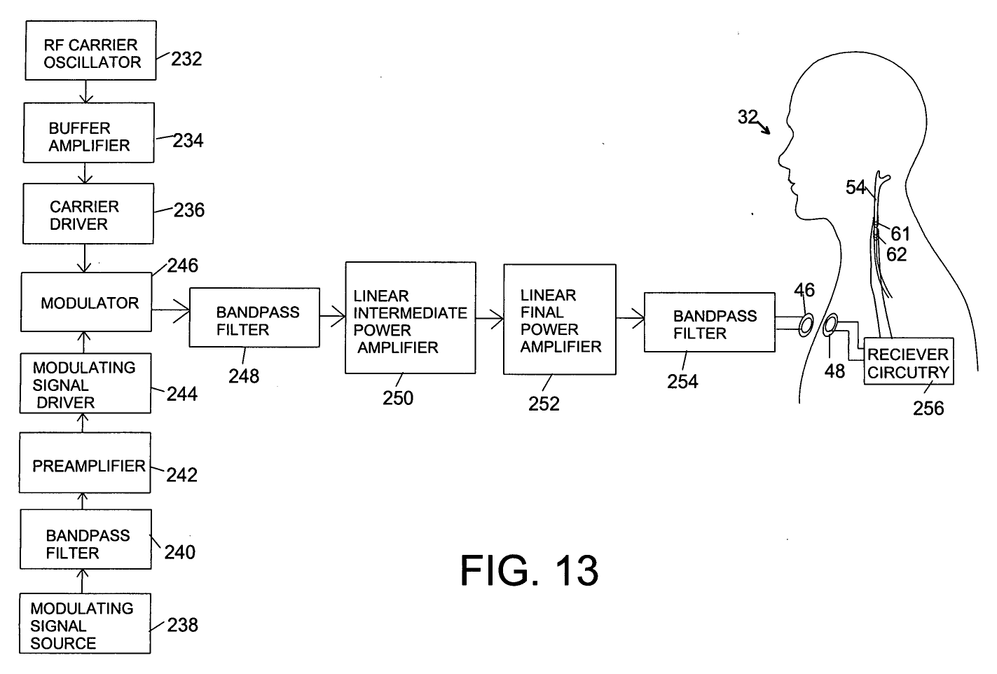

For utilizing an external power source, a passive implanted stimulus-receiver may be used. This embodiment of the vagus nerve pulse generator means is shown in conjunction with FIG. 13. A modulator 246 receives analog (sine wave) high frequency “carrier” signal and modulating signal. The modulating signal can be multilevel digital, binary, or even an analog signal. In this embodiment, mostly multilevel digital type modulating signals are used. The modulated signal is amplified 250, 252, conditioned 254, and transmitted via a primary coil 46 which is external to the body. A secondary coil 48 of an implanted stimulus receiver, receives, demodulates, and delivers these pulses to the vagus nerve(s) 54 via electrodes 61 and 62. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

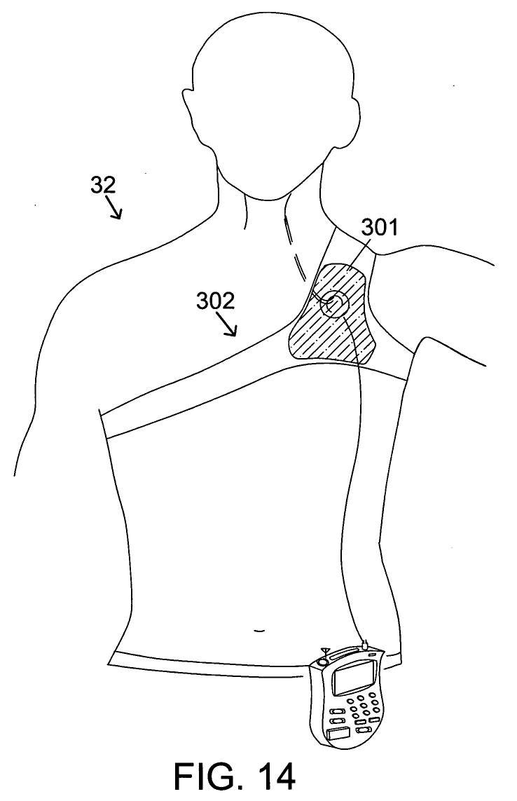

Shown in conjunction with FIG. 14, the coil for the external transmitter (primary coil 46) may be placed in the pocket 301 of a customized garment 302, for patient convenience.

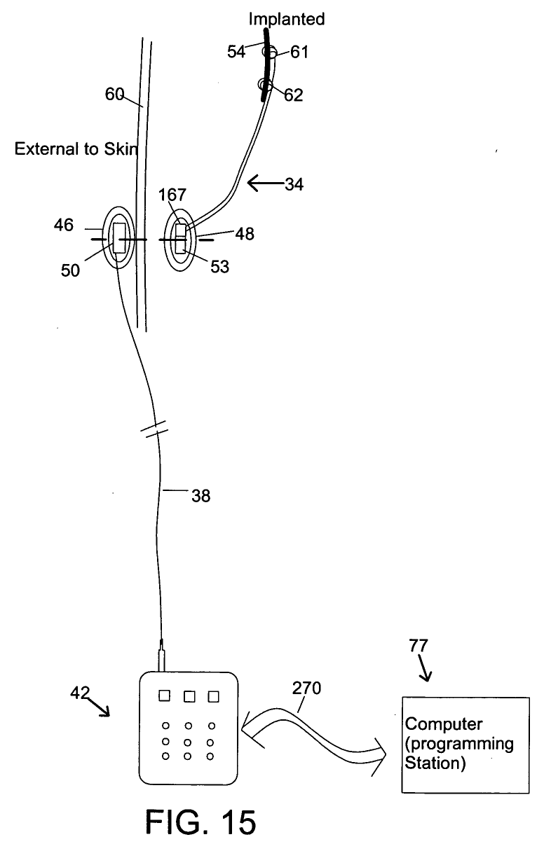

Shown in conjunction with FIG. 15, the primary (external) coil 46 of the external stimulator 42 is inductively coupled to the secondary (implanted) coil 48 of the implanted stimulus-receiver 34. The implantable stimulus-receiver 34 has circuitry at the proximal end, and has two stimulating electrodes at the distal end 61,62. The negative electrode (cathode) 61 is positioned towards the brain and the positive electrode (anode) 62 is positioned away from the brain.

For therapy to commence, the primary (external) coil 46 is placed on the skin 60 on top of the surgically implanted (secondary) coil 48. An adhesive tape may be placed on the skin 60 and external coil 46 such that the external coil 46, is taped to the skin 60. For efficient energy transfer to occur, it is important that the primary (external) 46 and secondary (internal) coils 48 be positioned along the same axis and be optimally positioned relative to each other. In this embodiment, the external coil 46 may be connected to proximity sensing circuitry 50, in which case the correct positioning of the external coil 46 with respect to the internal coil 48 is indicated by turning “on” of a light emitting diode (LED) on the external stimulator 42.

The programmable parameters are stored in a programmable logic in the external stimulator 42. The predetermined programs stored in the external stimulator 42 are capable of being modified through the use of a separate programming station 77. A Programmable Array Logic Unit and interface unit are interfaced to the programming station 77. The programming station 77 can be used to load new programs, change the existing predetermined programs or the program parameters for various stimulation programs. The programming station is connected to the programmable array unit (comprising programmable array logic and interface unit) with an RS232-C serial connection. The main purpose of the serial line interface is to provide an RS232-C standard interface. Other well known interface connections may also be used.

This method enables any portable computer with a serial interface to communicate and program the parameters for storing the various programs. The serial communication interface receives the serial data, buffers this data and converts it to a 16 bit parallel data. The programmable array logic component of programmable array unit (not shown) receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

The selective stimulation of the vagus nerve(s) can be performed in one of two ways. One method is to activate one of several “pre-determined/pre-packaged” programs. A second method is to “custom” program the electrical parameters, which can be selectively programmed for specific therapy to the individual patient. The electrical parameters that can be individually programmed, include variables such as pulse amplitude, pulse width, frequency of stimulation, stimulation on-time, and stimulation off-time. Table one below defines the approximate range of parameters,

TABLE 1
Electrical parameter range delivered to the nerve

| PARAMER | RANGE |
|---|---|
| Pulse Amplitude | 0.1 Volt-10 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 1 are the electrical signals delivered to the nerve via the two electrodes 61,62 (distal and proximal) around the nerve, as shown in FIG. 15. It being understood that the signals generated by the external pulse generator 42 and transmitted via the primary coil 46 are larger, because the attenuation factor between the primary coil 46 and secondary coil 48 is approximately 10-20 times, depending upon coupling factors such as the distance, and orientation between the two coils. Accordingly, the range of transmitted signals of the external stimulator 42 are approximately 10-20 times larger than shown in Table 1.

Referring to FIG. 16, the implanted lead component of the system is similar to cardiac pacemaker leads, except for distal portion (or electrode end) of the lead. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table two below.

TABLE 2
Lead design variables

| Proximal | Distal | | | | |
|---|---|---|---|---|---|
| End | End | | | | |
| | | | Conductor | | |
| | | | (connecting | | |
| | Lead body- | | proximal | | |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Spiral |
| bipolar | | coating | Nickel- | Platinum | electrode |
| | | | Cobalt | | |
| Bifurcated | Silicone | Anti- | | Platinum- | Wrap-around |
| | | Inflammatory | | Iridium | electrode |
| | | coating | | (Pt/lr) Alloy | |
| | Silicone with | Lubricious | | Pt/lr coated | Steroid |
| | Polytetrafluoro- | coating | | with Titanium | eluting |
| | ethylene | | | Nitride | |
| | (PTFE) | | | | |
| | | | | Carbon | Hydrogel |
| | | | | | electrodes |
| | | | | | Cuff |
| | | | | | electrodes |

Once the lead is fabricated, coating such as anti-microbial, anti-inflammatory, or lubricious coating may be applied to the lead body 59.

### Implanted Stimulus-Receiver Comprising a High Value Capacitor for Storing Charge, Used in Conjunction with an External Stimulator

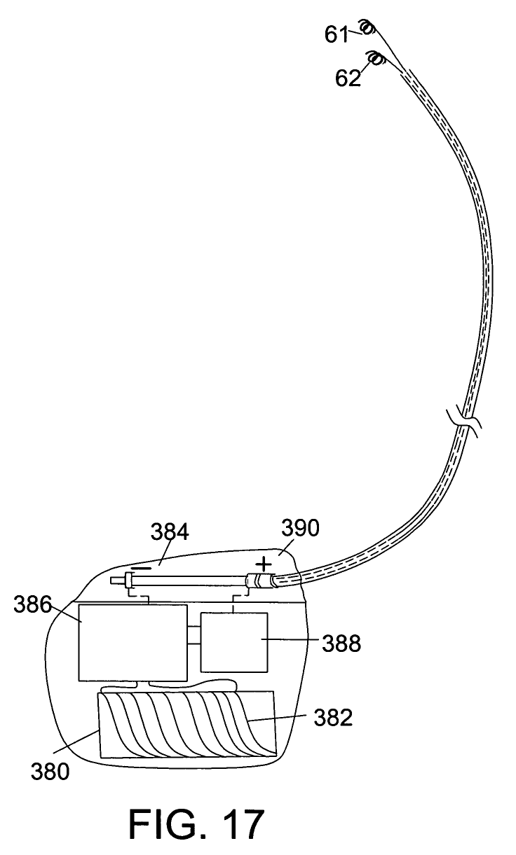

In one embodiment, the implanted stimulus-receiver may be a system which is RF coupled combined with a power source. In this embodiment, the implanted stimulus-receiver comprises high value, small sized capacitor(s) for storing charge and delivering electric stimulation pulses for up to several hours by itself, once the capacitors are charged. The packaging is shown in FIG. 17. Using mostly hybrid components and appropriate packaging, the implanted portion of the system described below can be miniaturized. As shown in FIG. 17, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment, as was shown previously in FIG. 14.

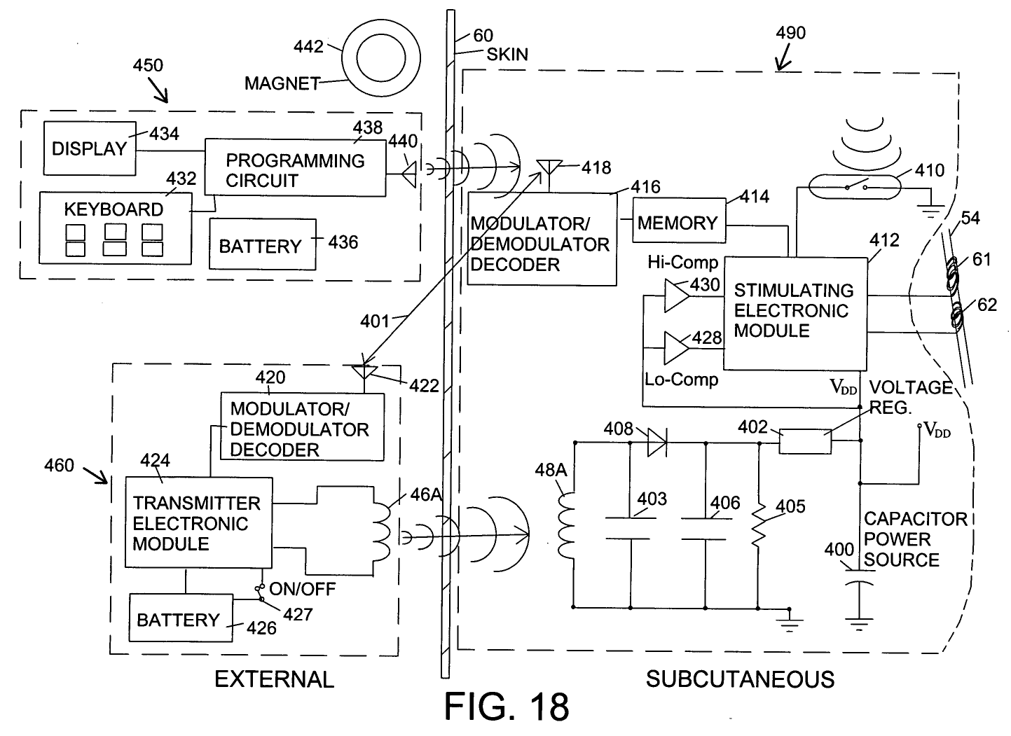

As shown in conjunction with FIG. 18 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V₁ fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 424, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 424 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve(s) 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic-module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

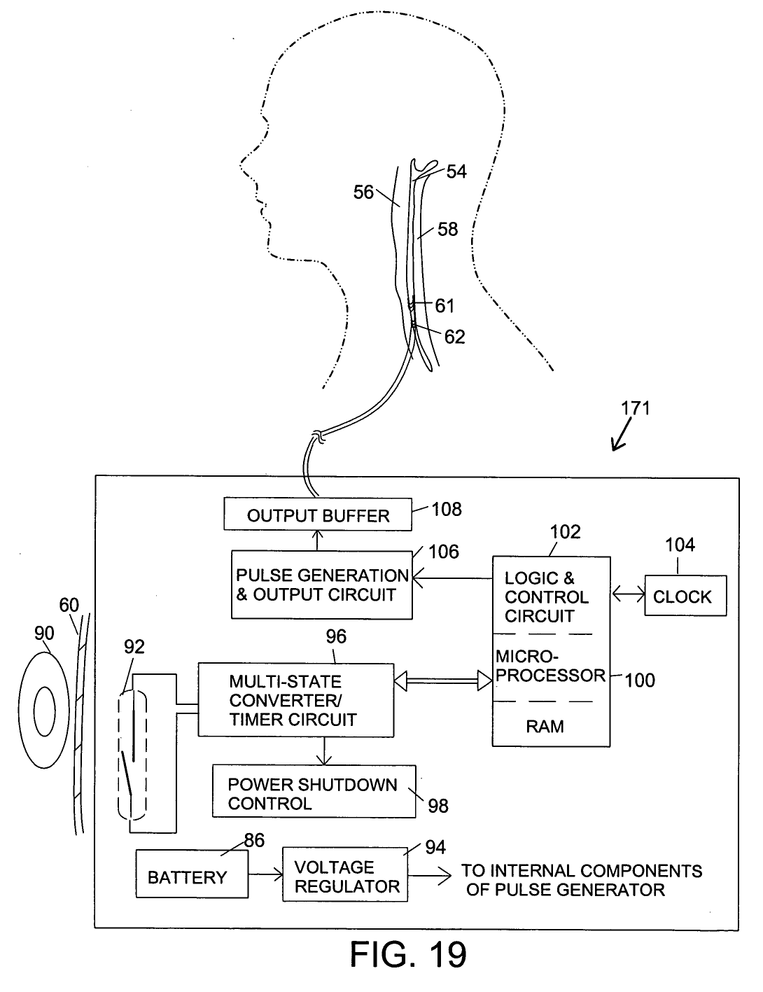

In one embodiment, a programmer-less implantable pulse generator (IPG) may be used. In this embodiment, shown in conjunction with FIG. 19, the implantable pulse generator 171 is provided with a reed switch 92 and memory & control circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

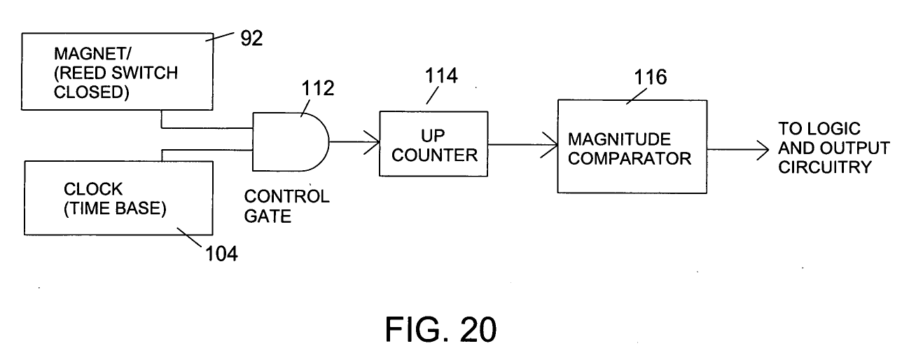

In one embodiment, shown in conjunction with FIG. 20, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, the pulse generation and amplification circuit 106 deliver the appropriate electrical pulses to the vagus nerve(s) 54 of the patient via an output buffer 108 (as shown in FIG. 19). The delivery of output pulses is configured such that the distal electrode 61 (electrode closer to the brain) is the cathode, and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,

| | |
|---|---|
| Current output: | 0.75 milliAmps. |
| Pulse width: | 0.20 msec. |
| Pulse frequency: | 20 Hz |
| Cycles: | 20 sec. on-time and 2.0 min. off-time |
| | in repeating cycles. |

LOW-MED stimulation state example is,

| | |
|---|---|
| Current output: | 1.5 milliAmps, |
| Pulse width: | 0.30 msec. |
| Pulse frequency: | 25 Hz |
| Cycles: | 1.5 min. on-time and 20.0 min. off-time |
| | in repeating cycles. |

MED stimulation state example is,

| | |
|---|---|
| Current output: | 2.0 milliAmps. |
| Pulse width: | 0.30 msec. |
| Pulse frequency: | 30 Hz |
| Cycles: | 1.5 min. on-time and 20.0 min. off-time |
| | in repeating cycles. |

HIGH stimulation state example is,

| | |
|---|---|
| Current output: | 3.0 milliAmps, |
| Pulse width: | 0.40 msec. |
| Pulse frequency: | 30 Hz |
| Cycles: | 2.0 min. on-time and 20.0 min. off-time |
| | in repeating cycles. |

These prepackaged/predetermined programs are mearly examples, and the actual stimulation parameters will deviate from these depending on the patient or treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet 90 triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, turns the device OFF.

The advantage of this embodiment is that it is cheaper to manufacture than a fully programmable implantable pulse generator (IPG).

### Programmable Implantable Pulse Generator (IPG)

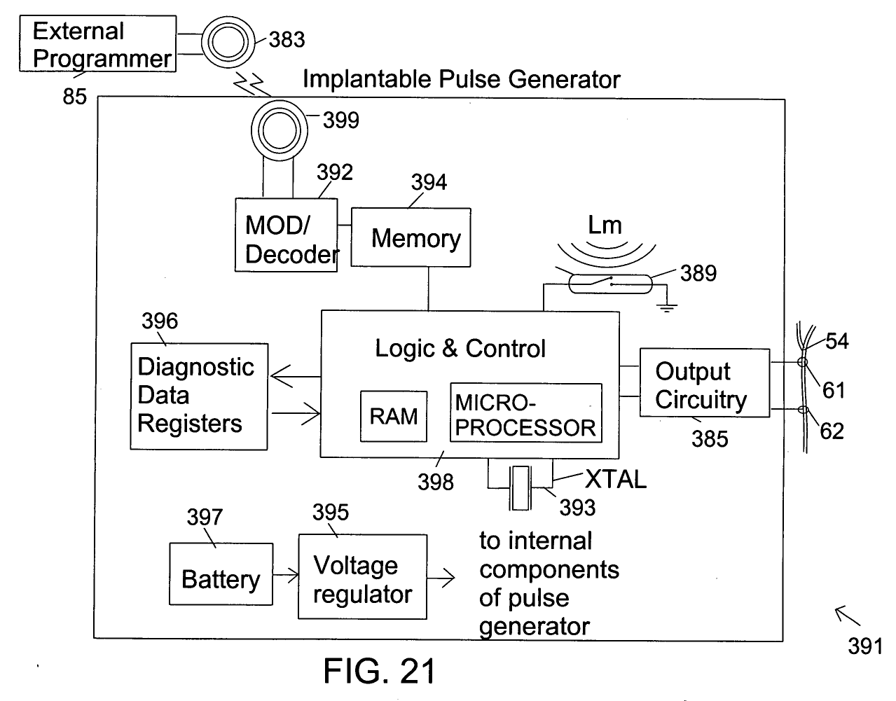

In one embodiment, a fully programmable implantable pulse generator (IPG) may be used. Shown in conjunction with FIG. 21, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40 (not shown). Programming of the implantable pulse generator (IPG) is done via an external programmer 85. Once programmed via an external programmer 85, the implanted pulse generator 391 provides appropriate electrical stimulation pulses to the vagus nerve(s) 54 via electrodes 61,62.

This embodiment may also comprise optional fixed pre-determined/pre-packaged programs. Examples of LOW, LOW-MED, MED, and HIGH stimulation states were given in the previous section, under “Programmer-less Implantable Pulse Generator (IPG)”. These pre-packaged/pre-determined programs comprise unique combinations of pulse amplitude, pulse width, pulse frequency, ON-time and OFF-time. Advantageously, a number of these “pre-determined/pre-packaged programs” may be stored in a “library”, and activated in a simple fashion, without having to program each parameter individually.

In addition, each parameter may be individually programmed and stored in memory. The range of programmable electrical stimulation parameters are shown in table 3 below.

TABLE 3
Programmable electrical parameter range

| PARAMER | RANGE |
|---|---|
| Pulse Amplitude | 0.1 Volt-10 Volts |
| Pulse width | 20 μS-5 mSec. |
| Frequency | 3 Hz-300 Hz |
| On-time | 5 Secs-24 hours |
| Off-time | 5 Secs-24 hours |
| Ramp | ON/OFF |

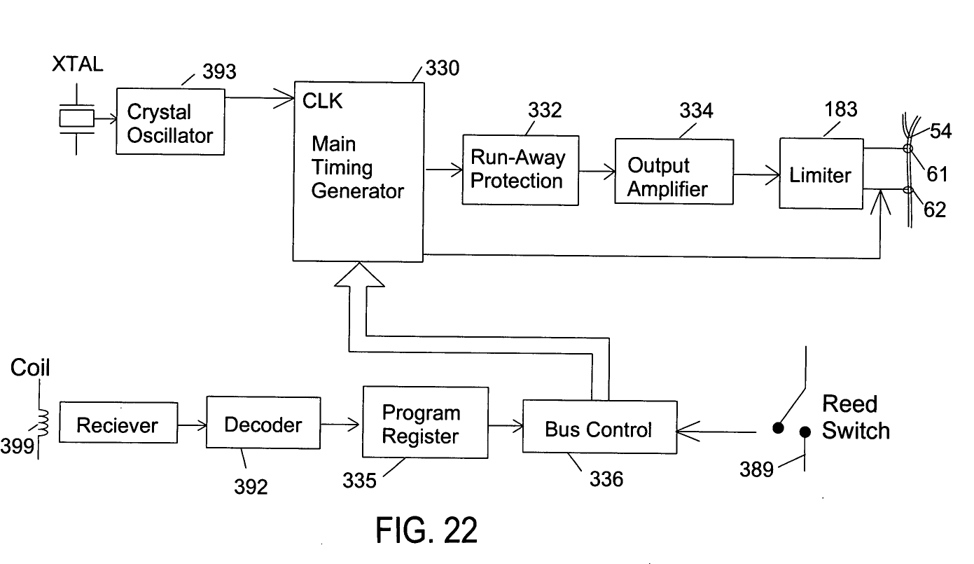

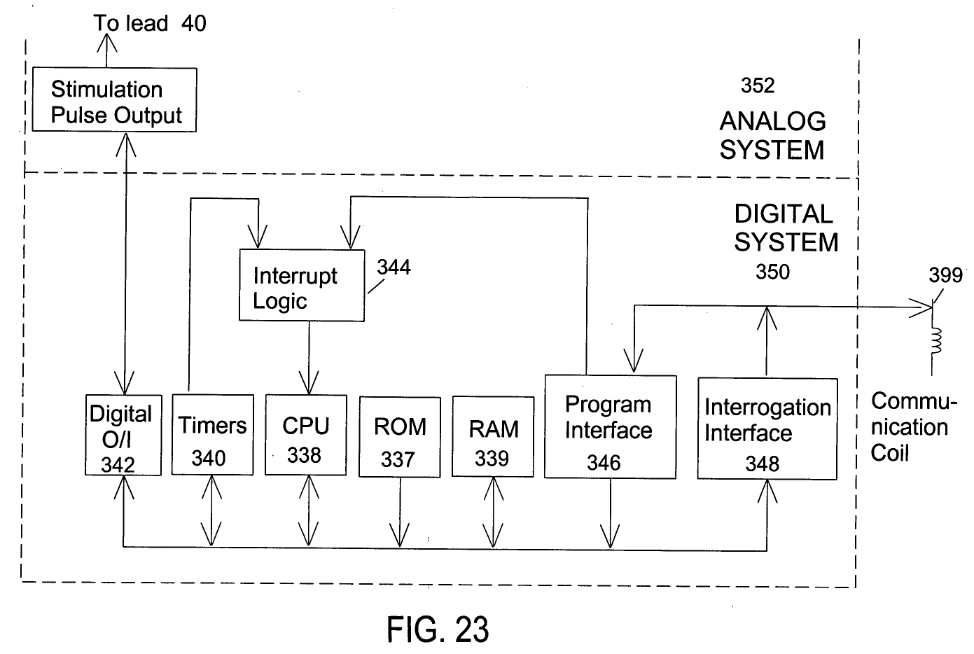

Shown in conjunction with FIGS. 22 and 23, the electronic stimulation module comprises both digital 350 and analog 352 circuits. A main timing generator 330 (shown in FIG. 22), controls the timing of the analog output circuitry for delivering neuromodulating pulses to the vagus nerve(s) 54, via output amplifier 334. Limiter 183 prevents excessive stimulation energy from getting into the vagus nerve(s) 54. The main timing generator 330 receiving clock pulses from crystal oscillator 393. Main timing generator 330 also receiving input from programmer 85 via coil 399. FIG. 23 highlights other portions of the digital system such as CPU 338, ROM 337, RAM 339, program interface 346, interrogation interface 348, timers 340, and digital O/I 342. The functioning details of these circuits is well known to one skilled in the art.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

### Combination Implantable Device Comprising Both a Stimulus-Receiver and a Programmable Implantable Pulse Generator (IPG)

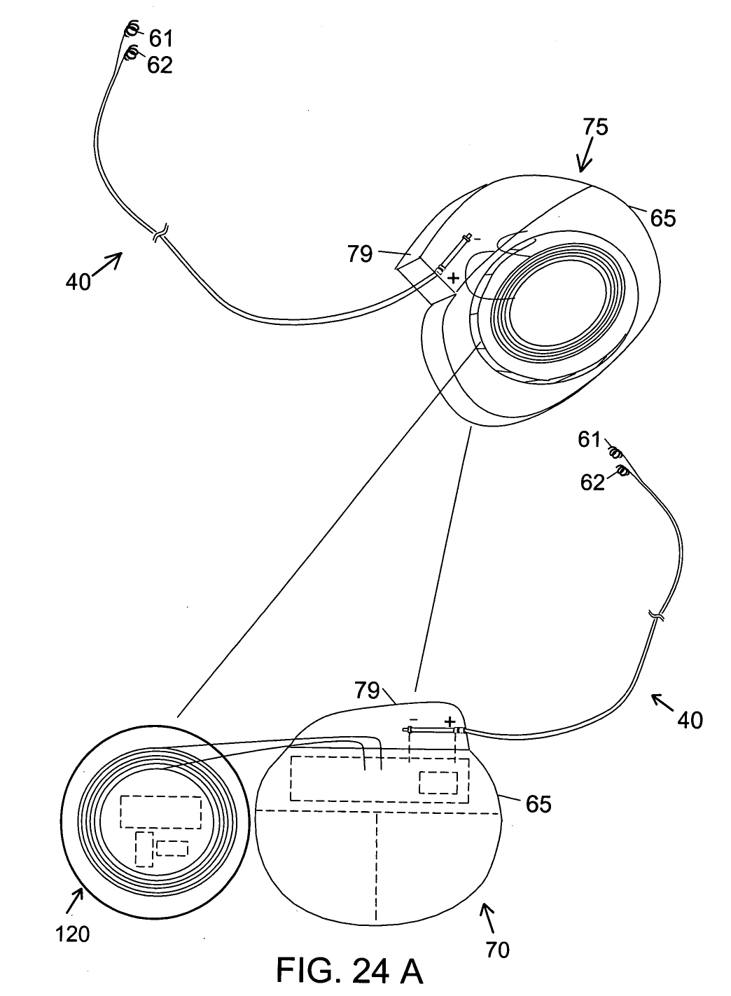

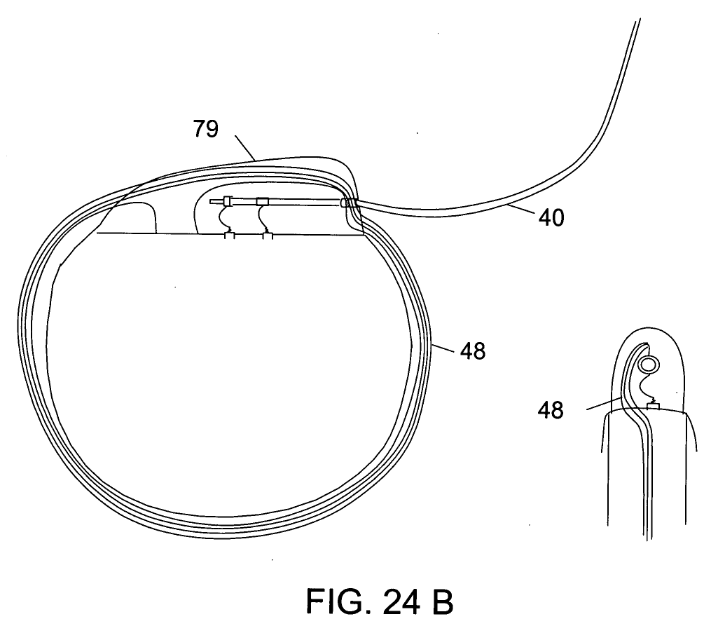

In one embodiment, the implantable device may comprise both a stimulus-receiver and a programmable implantable pulse generator (IPG). FIG. 24 shows a close up view of the packaging of the implanted stimulator 75 of this embodiment, showing the two subassemblies 120, 70. The two subassemblies are the stimulus-receiver module 120 and the battery operated pulse generator module 70. The electrical components of the stimulus receiver module 120 may be substantially in the titanium case along with other circuitry, except for a coil. The coil may be outside the titanium case as shown in FIG. 24A or may be around the titanium case as shown in FIG. 24B. The external stimulator 42, and programmer 85 also being remotely controllable from a distant location via the internet. Controlling circuitry means within the stimulator 75, makes the inductively coupled stimulator 120 and the IPG 70 operate in harmony with each other. For example, when stimulation is applied via the inductively coupled system, the battery operated portion of the stimulator is triggered to go into the “sleep” mode. Conversely, when programming pulses (which are also inductively coupled) are being applied to the implanted battery operated pulse generator 70, the inductively coupled stimulation circuitry 120 is disconnected.

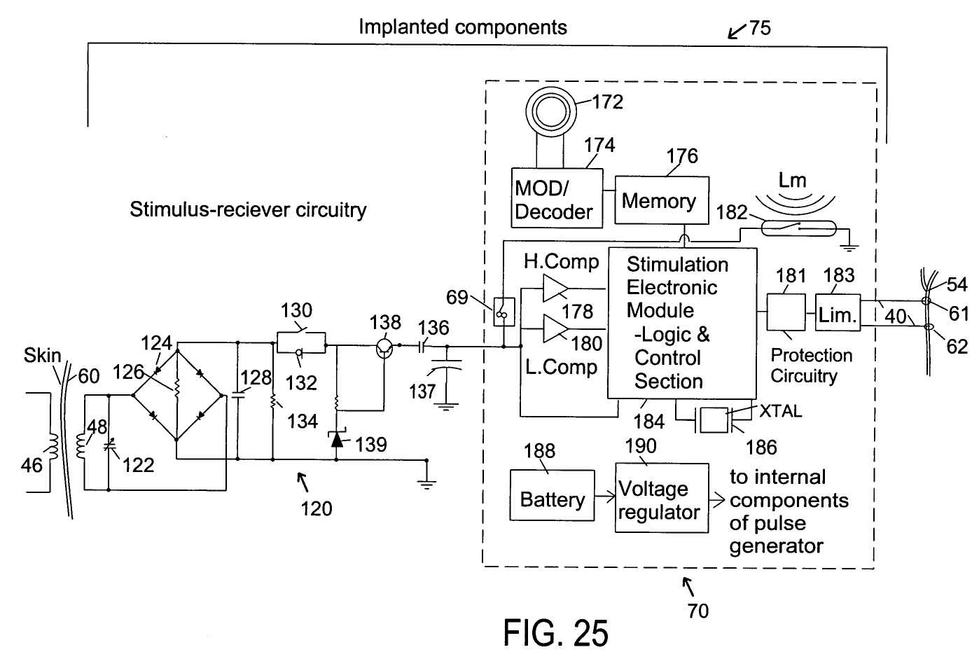

Shown in conjunction with FIG. 25, in one aspect of control circuitry, to program the implanted portion of the stimulator 70, a magnet is placed over the implanted pulse generator 70, causing a magnetically controlled Reed Switch 182 (which is normally in the open position) to be closed, at the same time a switch going to the stimulator lead 40, and a switch 69 going to the circuit of the stimulus-receiver module 120 are both opened, disconnecting both subassemblies electrically. Further, protection circuitry 181 is an additional safeguard for inadvertent leakage of electrical energy into the nerve tissue 54 during programming. Alternatively, instead of a reed switch 182, a solid state magnet sensor (Hall-effect sensor) may be used for the same purpose. The solid-state magnet sensor is preferred, since there are no moving parts that can get stuck.

With reference to FIG. 25, for the functioning of the inductively coupled stimulus-receiver 120, a primary (external) coil 46 is placed in close proximity to secondary (implanted) coil 48. The primary coil 46 may be taped to skin 60, or other means may be used for keeping the primary coil 46 in close proximity to the implanted (secondary) coil 48. Referring to the left portion of FIG. 25, the amplitude and pulse width modulated radiofrequency signals from the primary (external) coil 46 are inductively coupled to the secondary (implanted) coil 48 in the implanted unit 75. The two coils 46 and 48 thus act like an air-gap transformer. The system having means for proximity sensing between the two coils 46, 48, and feedback regulation of signals as described in a co-pending application.

Again with reference to FIG. 25, the combination of capacitor 122 and inductor 48 tunes the receiver circuitry to the high frequency of the transmitter with the capacitor 122. The receiver is made sensitive to frequencies near the resonant frequency of the tuned circuit, and less sensitive to frequencies away from the resonant frequency. A diode bridge 124 rectifies the alternating voltages. Capacitor 128 and resistor 134 filter out the high-frequency component of the receiver signal, and leaves the current pulse of the same duration as the bursts of the high-frequency signal. A zenor diode 139 is used for regulation and capacitor 136 blocks any net direct current.

As shown in conjunction with FIG. 25 the pulses generated from the stimulus-receiver circuitry 120 are compared to a reference voltage, which is programmed in the implanted pulse generator 70. When the voltage of incoming pulses exceeds the reference voltage, the output of the comparator 178,180 sends digital pulse 89 to the stimulation electric module 184. At this predetermined level, the high threshold comparator 178 fires and the controller 184 suspends any stimulation from the implanted pulse generator 70. The implanted pulse generator 70 goes into “sleep” mode for a predetermined period of time. In one preferred embodiment, the level of voltage needed for the battery operated stimulator to go into “sleep” mode is a programmable parameter. The length of time, the implanted pulse generator 70 remains in “sleep” mode is also a programmable parameter. Therefore, advantageously the external stimulator 42 in conjunction with the inductively coupled part of the stimulator 120 can be used to save the battery life of the implanted stimulator 75. It will be clear to one skilled in the art, that even though an analog implementation of the control circuitry is shown here, with some modifications digital implementations of control circuitry can readily be accomplished. Further, the stimulus-receiver coil 48 and the telemetry coil 172 can be combined into the same coil, which may be outside of the titanium can, as was shown in FIG. 24B.

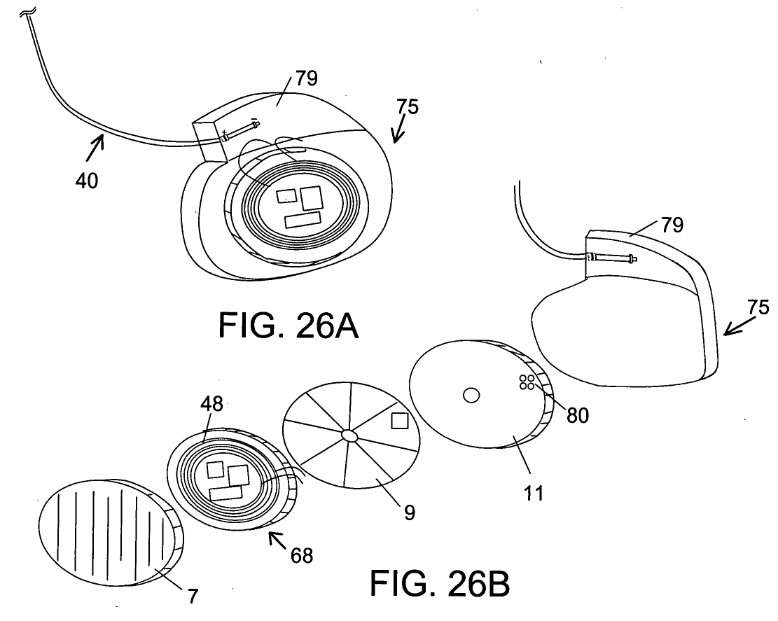

FIG. 26A shows a diagram of one embodiment of the finished implantable stimulator 75. FIG. 26B shows the pulse generator with some of the components used in assembly in an exploded view. These components include a coil cover 7, the secondary coil 48 and associated components, a magnetic shield 9, and a coil assembly carrier 11. The coil assembly carrier 11 has at least one positioning detail 13 located between the coil assembly and the feed through for positioning the electrical connection. The positioning detail 13 secures the electrical connection.

### Implantable Pulse Generator (IPG) Comprising a Rechargable Battery

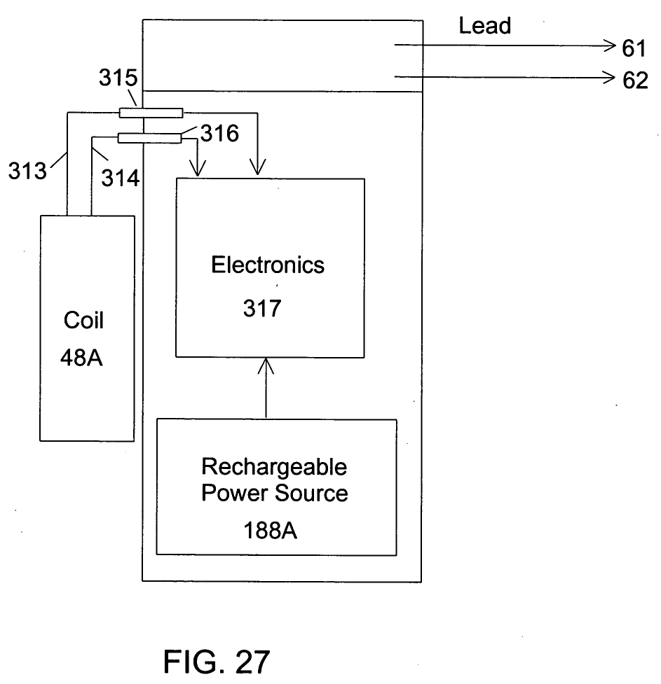

In one embodiment, an implantable pulse generator with rechargeable power source can be used. In such an embodiment (shown in conjunction with FIG. 27), a recharge coil 48A is external to the pulse generator titanium can. The RF pulses transmitted via an external coil 46 and received via subcutaneous coil 48A are rectified via diode bridge. These DC pulses are processed and the resulting current applied to recharge the battery 188A in the implanted pulse generator.

In summary, in the method of the current invention for neuromodulation of cranial nerve such as the vagus nerve(s), to provide therapy for psychiatric disorders, neuropsychiatric disorders and cognitive impairments, can be practiced with any of the several pulse generator systems disclosed including,
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a programmable implantable pulse generator;
e) a combination implantable device comprising both a stimulus-receiver and a programmable IPG; and
f) an IPG comprising a rechargeable battery.

Neuromodulation of vagus nerve(s) with any of these systems is considered within the scope of this invention.

In one embodiment, the external stimulator and/or the programmer has a telecommunications module, as described in a co-pending application, and summarized here for reader convenience. The telecommunications module has two-way communications capabilities.

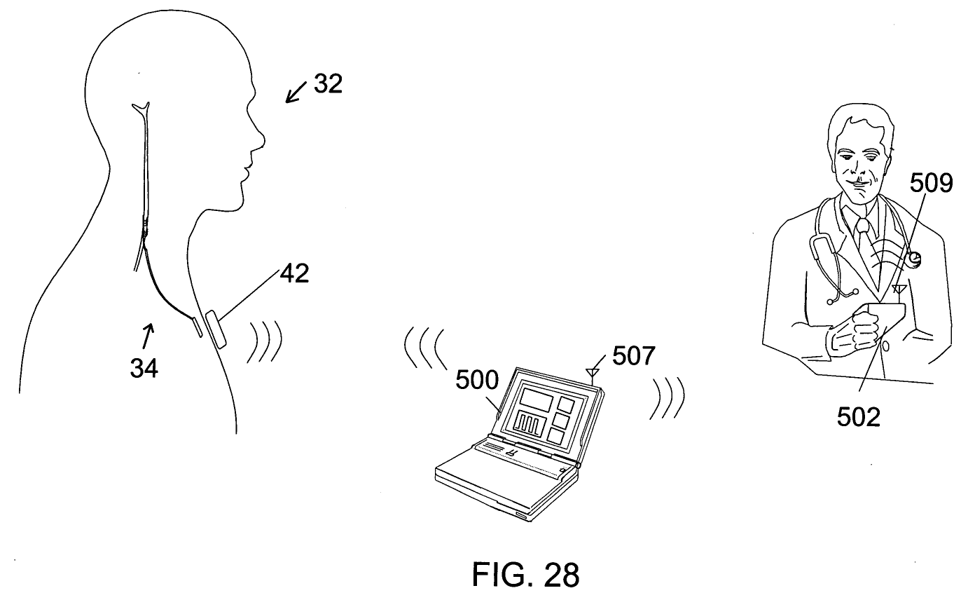

FIG. 28 depicts communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, CA) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

In one aspect of the invention, the telecommunications component can use Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

In one aspect, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device 42 to download these parameters.

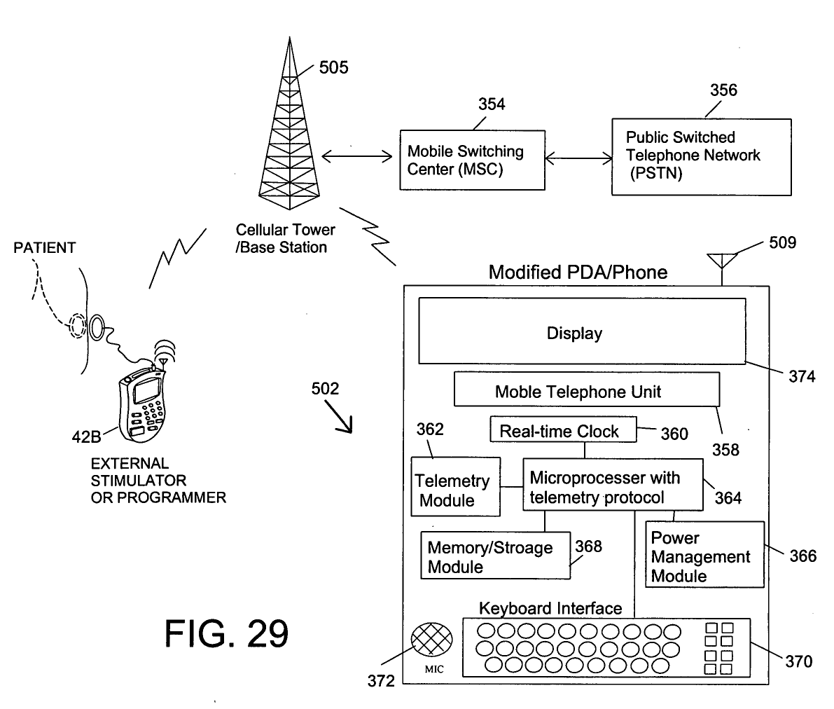

Shown in conjunction with FIG. 29 the physician's remote communication's module is a Modified PDA/Phone 502 in this embodiment. The Modified PDA/Phone 502 is a microprocessor based device as shown in a simplified block diagram in FIG. 29. The PDA/Phone 502 is configured to accept PCM/CIA cards specially configured to fulfill the role of communication module of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

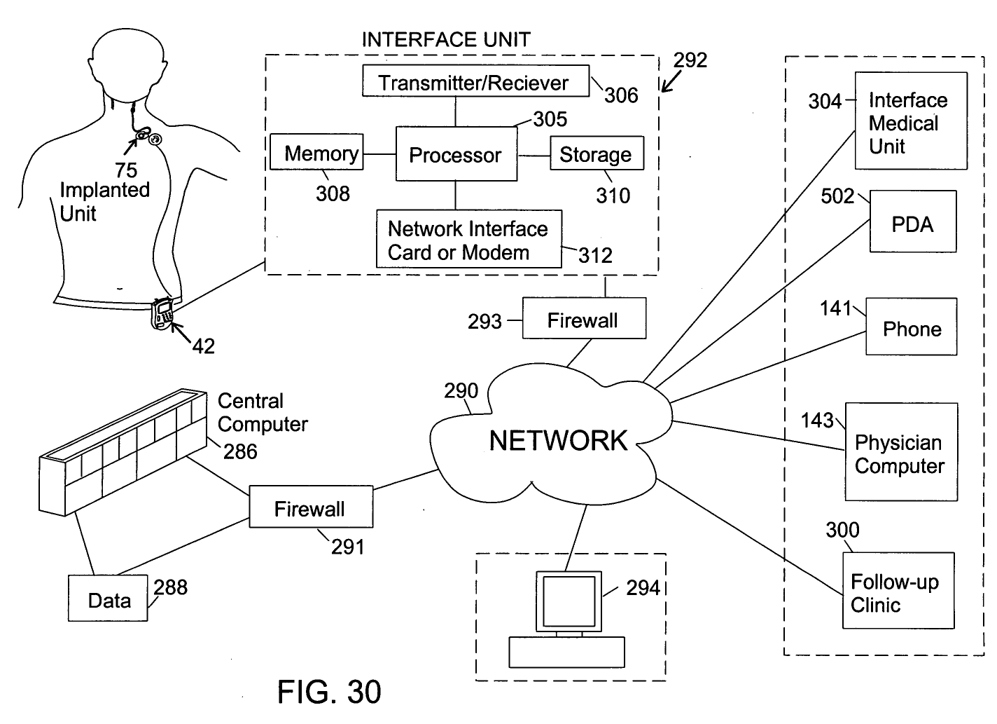

Shown in conjunction with FIG. 30, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit 75.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use “3 G” or above versions of technology for wireless communication and data exchange, even though in some cases “2.5 G” is being used currently.

For the system of the current invention, the use of any of the “3 G” technologies for communication for the Modified PDA/Phone 502, is considered within the scope of the invention. Further, it will be evident to one of ordinary skill in the art that as future “4 G” systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

1. A method of treating or alleviating the symptoms of neuropsychiatric disorders and cognitive impairments using gradient magnetic pulses to the brain and pulsed electrical stimulation to vagus nerve(s), comprising the steps of:
a) selecting a patient for providing said therapy;
b) providing gradient magnetic pulses to the brain of said patient; and
c) providing pulsed electrical stimulation to a vagus nerve(s) of said patient.

2. The method of claim 1, wherein said neuropsychiatric disorders and cognitive impairments further comprises depression, bipolar depression, anxiety disorders, obsessive-compulsive disorders, schizophrenia, borderline personality disorders, sleep disorders, learning difficulties, and memory impairments.

3. The method of claim 1, wherein said gradient magnetic pulses have a frequency of about 1 kHz and produce electric fields of the same frequency.

4. The method of claim 3, wherein said second electric fields have an amplitude of approximately between 1 V/m and 100 V/m.

5. The method of claim 3, wherein said electric fields have a duration of about 10 milliseconds.

6. The method of claim 1, wherein said gradient magnetic pulses to the brain are provided by a magnetic resonance spectroscopic imaging system.

7. The method of claim 1, wherein said pulsed electrical stimulation to vagus nerve(s) is by provided at any point along the length of said vagus nerve.

8. The method of claim 1, wherein said electric pulses to vagus nerve(s) are provided by at least one pulse generator from a group consisting of: an implanted stimulus-receiver with an external stimulator; an implanted stimulus-receiver comprising a high value capacitor for storing charge, used in conjunction with an external stimulator; a programmer-less implantable pulse generator (IPG) which is operable with a magnet; a programmable implantable pulse generator; a combination implantable device comprising both a stimulus-receiver and a programmable IPG; and an IPG comprising a rechargeable battery.

9. The method of claim 1, wherein said pulsed electrical stimulation to vagus nerve(s) is provided unilaterally or bilaterally.

10. The method of claim 1, wherein said gradient magnetic pulses provided to the brain and said electrical pulses provided to vagus nerve(s) are in any sequence, any combination or any time interval.

11. The method of claim 1, wherein said electric pulses provided to said vagus nerve(s) can be remotely controlled by wireless telemetry means.

12. A method of providing combination of gradient magnetic stimulation and pulsed electrical stimulation to a patient comprising, providing gradient magnetic pulses to the brain and electrical pulses to a vagus nerve(s) of said patient, whereby treating, or controlling, or alleviating the symptoms of neuropsychiatric disorders and cognitive impairments.

13. The method of claim 12, wherein said neuropsychiatric disorders and cognitive impairments further comprises depression, bipolar depression, anxiety disorders, obsessive-compulsive disorders, schizophrenia, borderline personality disorders, sleep disorders, learning difficulties, and memory impairments.

14. The method of claim 12, wherein said electrical pulses are provided at any point along the length of said vagus nerve(s).

15. The method of claim 12, wherein said electric pulses are provided by at least one pulse generator from a group consisting of: an implanted stimulus-receiver with an external stimulator; an implanted stimulus-receiver comprising a high value capacitor for storing charge, used in conjunction with an external high value capacitor for storing charge, used in conjunction with an external stimulator; a programmer-less implantable pulse generator (IPG) which is operable with a magnet; a programmable implantable pulse generator; a combination implantable device comprising both a stimulus-receiver and a programmable IPG; and an IPG comprising a rechargeable battery.

16. The method of claim 12, wherein said vagus nerve(s) stimulation is unilateral or bilateral.

17. The method of claim 12, wherein said gradient magnetic pulses to the brain are provided by a magnetic resonance spectroscopic imaging system.

18. The method of claim 12, wherein said gradient magnetic pulses have a frequency of about 1 kHz and produce electric fields of the same frequency.

19. The method of claim 18, wherein said electric fields have a duration of about 10 milliseconds.

20. The method of claim 12, wherein said second electric fields have an amplitude of approximately between 1 V/m and 100 V/m.

21. The method of claim 12, wherein said combination of providing gradient magnetic pulses to the brain and electrical pulses to vagus nerve(s) of a patient is in any sequence or time interval.

22. The method of claim 12, wherein said electric pulses to said vagus nerve(s) are remotely controllable by wireless telemetry means.

23. A system for treating or controlling or alleviating the symptoms of neuropsychiatric disorders, and cognitive impairments comprises,
a) means to provide gradient magnetic pulses to the brain of a patient, and
b) means to provide afferent neuromodulation of a vagus nerve(s) of said patient with pulsed electrical stimulation.

24. The system of claim 23, wherein said means to provide afferent neuromodulation of a vagus nerve(s) provides said neuromodulation by providing electric pulses at any point along the length of said vagus nerve(s).

25. The system of claim 23, wherein said means to provide afferent neuromodulation of vagus nerve(s) of a patient further comprises at least one pulse generator from a group comprising of: an implanted stimulus-receiver with an external stimulator; an implanted stimulus-receiver comprising a high value capacitor for storing charge, used in conjunction with an external stimulator; a programmer-less implantable pulse generator (IPG) which is operable with a magnet; a programmable implantable pulse generator; a combination implantable device comprising both a stimulus-receiver and a programmable IPG; and an IPG comprising a rechargeable battery.

26. The system of claim 23, wherein said vagus nerve(s) is/are neuromodulated by unilateral or bilateral stimulation.

27. The system of claim 23, wherein said means to provide gradient magnetic pulses to the brain further comprises a magnetic resonance spectroscopic imaging system.

28. The system of claim 23, wherein said gradient magnetic pulses have a frequency of about 1 kHz and produce electric fields of the same frequency.

29. The system of claim 28, wherein said electric fields have a duration of about 10 milliseconds.

30. The system of claim 28, wherein said electric fields have an amplitude of approximately between 1 V/m and 100 V/m.

31. The system of claim 23, wherein said neuropsychiatric disorders and cognitive impairments further comprises depression, bipolar depression, anxiety disorders, obsessive-compulsive disorders, schizophrenia, borderline personality disorders, sleep disorders, learning difficulties, and memory impairments.

32. The system of claim 23, wherein said means to provide gradient magnetic pulses to the brain and said means to provide afferent neuromodulation of the vagus nerve(s) in a patient are provided to said patient in any sequence or combination or time interval.

33. The system of claim 23, wherein said electric pulses to said vagus nerve are remotely controllable by wireless telemetry means.
